(19) United States
(12) Patent Application Publication
(10) Pub. No.: US 2011/0196454 A1
(43) Pub. Date: Aug. 11, 2011

(54) SENSING SYSTEM, DEVICE, AND METHOD FOR THERAPY MODULATION

(75) Inventors: Angela Strand, San Francisco, CA (US); Mark Zdeblick, Portola Valley, CA (US); George Savage, Portola valley, CA (US); David O'reilly, Palo Alto, CA (US)

(73) Assignee: PROTEUS BIOMEDICAL, INC., Redwood City, CA (US)

(21) Appl. No.: 12/665,695

(22) PCT Filed: Nov. 13, 2009
(86) PCT No.: PCT/US2009/064427

§ 371 Date: Dec. 18, 2009

Related U.S. Application Data
(60) Provisional application No. 61/115,902, filed on Nov. 18, 2008.

(51) Int. Cl. A61N 1/36

(52) U.S. Cl. 607/62

(57) ABSTRACT

Sensing system, device, and method for therapy modulation are provided. Various aspects include a receive module and a therapy-related action module. The receive module may receive a therapy parameter of an individual. The therapy-related action module may effectuate, based on the therapy parameter, a therapy-related action associated with a nervous system of the individual.

### CROSS-REFERENCE TO RELATED APPLICATIONS

Pursuant to 35 U.S.C. § 119 (e), this application claims priority to the filing date of U.S. Provisional Patent Application Ser. No. 61/115,902 filed Nov. 18, 2008; the disclosures of which applications are herein incorporated by reference.

### INTRODUCTION

The mammalian nervous system comprises a network of specialized cells, i.e., neurons, which communicate information about an organism's surroundings and itself. The neurons of the nervous system are interconnected in complex arrangements and use electrochemical signals and neurotransmitters to transmit impulses from one neuron to the next. The interaction of the different neurons form neural circuits that regulate the organism's perception of the world and activities associated with its body, thus regulating its behavior.

Conditions related to the nervous system may result in chronic and/or acute pain sensations. Common chronic pain complaints include headache, low back pain, cancer pain, arthritis pain, neurogenic pain, and psychogenic pain. If a neural-related therapy, such as neural stimulation/modulation, were introduced to treat a neural condition, such as pain, the therapy may electrically target a fixed tissue site. While this approach may generally focus on a neural region associated with the chronic pain, the approach may not be predicated on a fully-informed basis with respect to fundamental data. As such a treatment may be generally applicable to a broad cross-section of individuals sharing common characteristics and condition parameter(s), the treatment, however, may not take into account the differing parameters or conditions specific to the individuals. Consequently, the treatment may not be able to meet the individual needs of a patient and optimize the therapy outcome of the patient.

### SUMMARY

Sensing methods for therapy modulation are provided. The methods include applying a therapy-related action based on a therapy parameter sensed or received. Also provided are systems and devices that implement the methods in a variety of applications using different components.

### BRIEF DESCRIPTION OF THE FIGURES

FIG. 1 illustrates a sensing system for therapy modulation in a patient therapy environment.

FIG. 2 illustrates the sensing system for therapy modulation of FIG. 1 in greater detail.

FIG. 3 illustrates a device for therapy modulation.

FIG. 4 illustrates a method for therapy modulation.

### DETAILED DESCRIPTION

Generally, the invention provides a sensing method, system, and device for therapy modulation. More particularly, the invention includes use of one or more therapy parameters to enhance therapy. Typically, the therapy involves neural-related treatment and/or treatment directed to a neural-related condition, e.g., therapy for pain management.

In various aspects, the therapy parameters, used alone or in conjunction with various algorithms, effectuate various therapy outcomes by, for example, informing therapy adjustments, stimulating via a therapy program, and automatically modulating therapy dimensions. In the latter, for example, the automatic modulation may be accomplished as a result of, or as a part of, a feedback system associated with the therapy parameter, e.g., a closed-loop feedback system based on the therapy parameter(s).

In various aspects, the therapy parameters may be tracked and stored. The tracked and stored data are sometimes referred to herein as “historical data”. The historical data may be employed for various purposes. For example, the historical data may be displayed as longitudinal information. The historical data may also be analyzed to determine an individual patient's “baseline” for therapy and response. Further, the historical data may be analyzed and modeled for effective program management, making recommendations, etc.

In various aspects, the therapy parameters may be used as bases for a modulated “tuning” program, i.e., a program that a patient, clinician, etc., may run on demand to intelligently modify the current therapy for enhanced results, etc. The tuning program, for example, may prompt the patient for feedback (various therapy parameters) and, based on the feedback, adjust the therapy. Other data, e.g., objective therapy parameters, may be incorporated for adjustment calculations.

Use of the invention may have many areas of applications which include neural stimulation and/or neuromodulation (sometimes collectively referred to herein as “neural stimulation”), cardiac rhythm management, etc. Certain aspects may include one or more sensors for sensing at least one therapy parameter. The sensors may be extra-corporeal, implanted, or both. The sensors may be communicably, e.g., electrically, mechanically, etc., associated with a device such as an external device or an implanted device. Examples of external devices include personal receivers, e.g., removably attachable receivers, such as those disclosed and described in PCT application serial nos. PCT/U.S. 2008/052845 published as WO/2008/095183 and PCT/U.S. 2006/016370 published as WO/2006/116718, the disclosures of which are herein incorporated by reference.

Certain aspects may be directed to in-body therapies and may include, for example, implantable medical devices. The term “implantable medical device”, as used herein, refers to a device configured to be positioned at least partially on a living body, at least partially in a living body, or a combination thereof. For example, the implantable medical device may include a lead having various electrode configurations communicably associated with controller circuitry, a power source, etc., and one or more sensors, e.g., a neural sensing, for sensing the therapy parameter.

More particularly and illustratively, the implantable medical device may comprise one or more leads with multiple in-line segmented electrode satellites, wherein each electrode is independently controllable and power/data wire(s) for multiplexing the multiple segmented electrode satellites. Various configurations of devices which may be used conjunction with this invention are described/disclosed in PCT application no. PCT/U.S. 2003/039524 published as WO 2004/052182; PCT application no. PCT/U.S. 2005/031559 published as WO 2006/029090; PCT application no. PCT/U.S. 2005/046811 published as WO 2006/069322; PCT application no. PCT/U.S. 2005/046815 published as WO 2006/069323; PCT application no. PCT/U.S. 2006/048944 published as WO 2007/075974; and U.S. application Ser. No. 11/939,524 published as U.S. 2008-0114230 A1, the disclosures of which are herein incorporated by reference.

Various configurations of devices which may be used conjunction with this invention are described/disclosed in PCT application serial no. PCT/U.S. 2006/016370 published as WO/2006/116718; PCT application serial no. PCT/U.S. 2007/082563 published as WO/2008/052136; PCT application serial no. PCT/U.S. 2007/024225 published as WO/2008/063626; PCT application serial no. PCT/U.S. 2007/022257 published as WO/2008/066617; PCT application serial no. PCT/U.S. 2008/052845 published as WO/2008/095183; PCT application serial no. PCT/U.S. 2008/053999 published as WO/2008/101107; PCT application serial no. PCT/U.S. 2008/056296 published as WO/2008/112577; and PCT application serial no. PCT/U.S. 2008/056299 published as WO/2008/112578; as well as U.S. Provisional Application Ser. Nos.: 61/114,433; 61/114,437; 61/115,887; 61/114,441; 61/114,442; and 61/114,443; the disclosures of which are herein incorporated by reference.

### Sensing System for Therapy Modulation

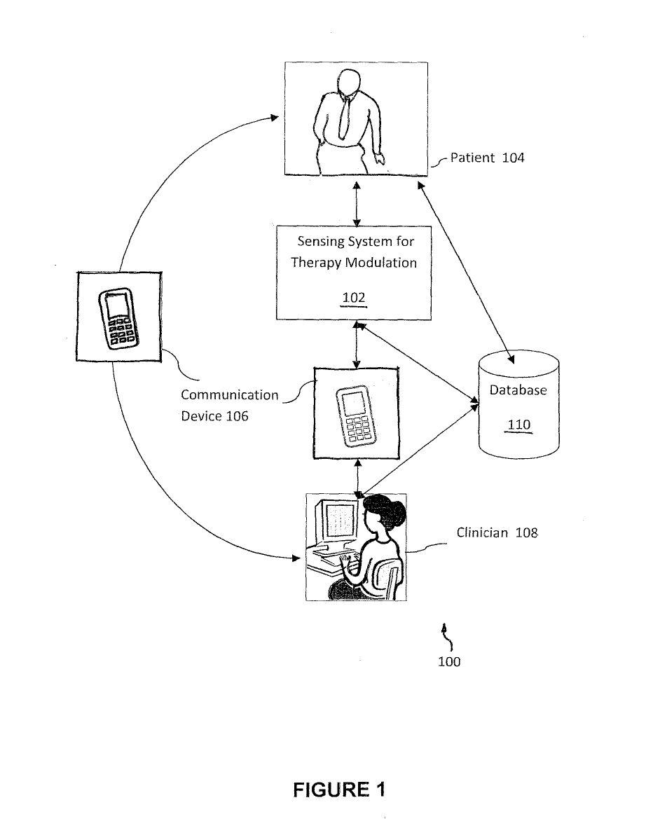

FIG. 1 illustrates a sensing system for therapy modulation 102 in a patient therapy environment 100. The sensing system for therapy modulation 102, sometimes referred to herein as “sensing system” 102, may be associated with, e.g., worn by, placed on, or implanted in, an individual, e.g., patient 104. The sensing system 102 may communicably interoperate, via various vehicles of communication, e.g., communication device(s) 106, channels, and /or modes, with one or more persons, e.g., clinician 108, and one or more storage media, e.g., with database 110, etc. The sensing system 102 may communicate via a variety of devices, modes, and combinations thereof, as discussed hereinafter.

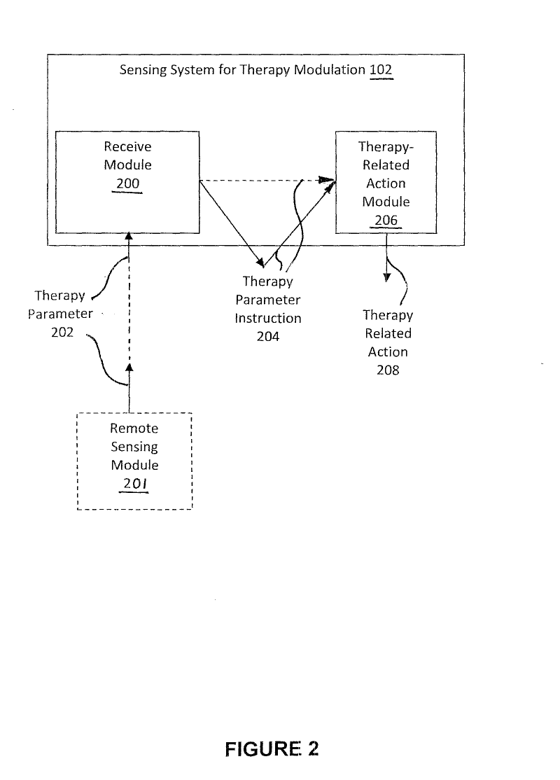

FIG. 2 illustrates the sensing system for therapy modulation 102 of FIG. 1 in greater detail. In various aspects, the sensing system 102 may comprise, for example, a receive module 200 to receive a therapy parameter 202 and to forward a therapy parameter instruction 204 to a therapy-related action module 206, which is communicably coupled to the receive module 200. The therapy-related action module 206 may effectuate, based on the therapy parameter instruction 204, a therapy-related action 208 associated with a patient's therapy. In various aspects, the sensing system 102 may further comprise a remote sensing module 201 which may be communicably coupled to the receive module 200 to sense the therapy parameter 202.

The receive module 200 may comprise any hardware component, software, and/or device, and combinations thereof, capable of carrying out the functionality of receiving the therapy parameter 202 and further capable of the functionality necessary to generate and/or communicate, directly or indirectly, the therapy parameter instruction 204 to the therapy-related action module 206. For example, in various aspects the receive module 200 may comprise circuitry, e.g., an integrated circuit, to receive a signal associated with the therapy parameter 202, and may further comprise a transmitter device to transmit the signal, directly or indirectly, to the therapy-related action module 206.

In one example implementation, the receive module 200 may comprise or be integrated into a lead having a receiver and circuitry logic for receiving and processing the therapy parameter 202 and, based on the therapy parameter 202, generating the therapy parameter instruction 204 for transmission to the therapy-related action module 206. The lead may further comprise multiple electrodes, e.g., sensing electrodes and stimulation electrodes. The lead may be implanted in an area associated with neural activity in the patient 104, and may be capable of providing stimulation to areas of neural tissue via one or more of the electrodes.

In another example implementation, the receive module 200 may have its associative sensing electrode(s) remotely, e.g., such as the remote sensing module 201. The remote sensing module 201 may comprise one or more hardware and/or software components and is capable of carrying out the functionality of sensing, generating, and/or communicating the therapy parameter 202. For example, the remote sensing module 201 may be a cardiac device implanted in the patient 104, such as a cardiac rhythm device, which senses or measures a heart parameter, such as a heart rate of the patient, and communicates the heart rate to the receive module 200. The receive module 200 may store, process, generate, transmit, etc. information containing the heart parameter for current and/or future informing purposes, etc. One example is to inform an algorithm or processing one or more steps employed in deciding the therapy parameter 202 or therapy parameter instruction 204.

The therapy parameter 202 may include any parameter associated with an individual, e.g., the patient 104, or an individual's therapy. The therapy parameter 202 may include various categories of parameters associated with the individual, e.g., a physiologic parameter, a subjective parameter, a chemical parameter, an electrical parameter, etc.

For example, the physiologic parameter may be heart rate, heart rate variability, an edema indicator, a fluid retention indicator, body temperature, a perspiration indicator, a galvanic skin response, blood pressure, a vasodilatation indicator, etc. The subjective parameter may be a patient-perceived pain indicator (PPPI), a patient-perceived tolerance indicator (PPTI), and a patient perceived-optimal treatment indicator (PPOTI). The chemical parameter may be an extracellular signal-regulated kinase (ERK), gamma-aminobutyric acid (GABA), acetylcholine (Ach), and a neural activity indicator. The electrical parameter may be an indicator of a conduction pattern associated with the nervous system or other electrical indicator.

The therapy parameter 202 may be generated and sensed via a variety of means and devices. For example, the physiologic parameter may be sensed via one or more sensors, such as the remote sensing module 201, embodied herein as cardiac sensing devices, fluid sensors, temperature sensors, chemical sensors, electrical sensors, etc. The subjective parameters may be generated/obtained via various communication devices such as mobile telephones having associated applications which facilitate the patient 104 to input various qualitative and/or quantitative therapy-parameters, e.g., the PPPI, the PPTI, the PPOTI, etc. As the patient 104 undergoes a pain-abatement therapy in the form of electrical stimulation(s) in a neural tissue region via the aforementioned lead, the patient may still be experiencing a certain level of pain in spite of the electrical stimulation. In such a case, the patient 104 may enter the PPPI, e.g., a scaled, numeric indicator of the patient's current perception of the degree or level of pain experienced. The PPPI may be entered, for example, using an application on the patient's mobile telephone or computer. The application then may forward the therapy parameter 202 to the receive module 200 to effect the therapy-related action module 206.

The therapy parameter instruction 204 may be used to derive data pertinent to the therapy-related action 208 to be taken. In various aspects, the therapy parameter instruction 204 comprises data, e.g., a data set, etc., communicated to and/or received by the therapy-related action module 206.

The therapy-related action module 206 may comprise any hardware component(s), software module(s), and/or device(s), and combinations thereof, capable of carrying out the functionality of receiving the therapy parameter instruction 204 and further capable of the functionality necessary to communicate, e.g., active or passive transfer, accessibility, etc. of the therapy parameter instruction 204 to effectuate the therapy-related action 208. For example, in various aspects, the therapy-related action module 206 may comprise circuitry, e.g., an integrated circuit, to receive a signal associated with the therapy parameter instruction 204, and may further comprise circuitry logic to carry out the therapy parameter instruction 204 to effectuate the therapy-related action 208.

In various aspects, the therapy-related action module 206 may be wholly or partially integrated, or otherwise associated with the receive module 200. In one example, the therapy-related action module 206 and the receive module 200 may be part of a lead or other therapy-related device. In various aspects, for example, circuitry on a device, e.g., a therapy device, may interpret the therapy parameter instruction 204 and cause the target of the therapy parameter instruction 204, e.g., the therapy device such as a neural stimulation device, to mechanically actuate a component to modulate therapy or to electrically effect one or more components of the therapy device to affect therapy, e.g., discontinue stimulation, increase intensity of the electrical field, adjust focus of an electrode, etc.

In various aspects, the therapy-related action module 206 may be independent of the receive module 200. In one example, the therapy-related action module 206 may be a part of the lead, but the receive module 200 may be implemented remote from the lead. The two devices may communicate, for example, via conduction associated with body fluid of the patient, radio frequency (RF) communications, or other means.

The therapy-related action 208, includes, initiating a therapy, modifying the therapy, stopping the therapy, discontinuing the therapy, informing the individual of the therapy, tracking the therapy parameter 202, storing the therapy parameter 202, displaying the therapy parameter 202, generating a baseline indication associated with the individual, e.g., the patient 104, displaying the baseline indication, generating an historical data model including the therapy parameter 202, determining a program based on at least one of the baseline indication and the historical data model, analyzing the therapy data, automatically recommending a therapy based on the analysis of the therapy data, automatically prompting the individual for feedback, and modifying the therapy based on the feedback.

In various aspects, at least one of the therapy parameter(s) 202, the therapy parameter instruction(s) 204, other data, or combinations thereof, may be stored/processed/displayed, etc., via various devices associated with, but not necessarily mechanically integrated with, at least one of the invention components.

In one example, a remote server having data storage and processing capabilities may utilize an application, e.g., the database 110 of FIG. 1, to interface with the system of the clinician 108, the communication device of the patient 104, or both. Such devices may facilitate various aspects of the present invention, e.g., the server may store, analyze, process, etc., various therapy-related data for longitudinal display via, for example, the system of the clinician 108 or the communication device of the patient 104.

In another example, patient-entered therapy parameters, e.g., the subjective parameter, may be communicated from a communication device associated with the patient 104 to the clinician's system, which may automatically (or with the clinician's intervention) process the received parameters in conjunction with, for example, various other patient data to generate a modulated treatment instruction, e.g., for automatic delivery via the patient's communication device to the therapy device, e.g., the lead. Upon receipt of the therapy parameter instruction 204, the lead's circuitry effectuates a control, e.g., an increase, decrease, or no change, in stimulation intensity of the lead to enhance the patient's therapy and lower the patient's pain perception.

### Device for Therapy Modulation

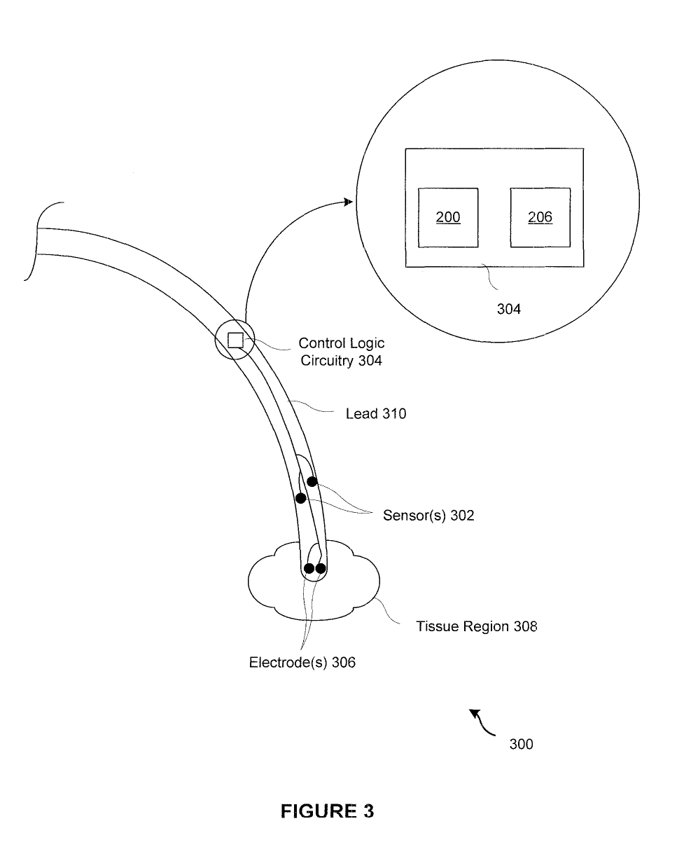

FIG. 3 illustrates a device for therapy modulation 300. The device for therapy modulation 300 comprises at least one sensor 302 to sense at least one therapy parameter, e.g., the therapy parameter 202 of FIG. 2, of an individual, such as the patient 104 of FIG. 1, and control logic circuitry 304 in electrical communication with the sensor 302 to receive the therapy parameter and to effect, based on the therapy parameter, a therapy-related action, e.g., the therapy-related action 208.

In various aspects, the device 300 is an implanted device, an implanted neural device, an extra-corporeal device, or a wearable device that can be actively-powered or passively-powered. In various aspects, the device 300 further comprises one or more electrodes 306 to stimulate a tissue region 308 associated with the individual. In various aspects, the electrodes 306 may be based on a network of electrodes.

In other aspects, the device 300 includes an integrated chip, which comprises the control logic circuitry 304, e.g., the receive module 200 and the therapy-related action module 206 of FIG. 2 in combination, implemented on a lead 310. The lead 310 also includes the electrodes 306 to stimulate the tissue region 308 and/or sense the therapy parameter, supra, as described in the incorporated reference(s). The lead 310 may be implanted in the patient to effect a neural region, e.g., stimulate neural tissue to conduct a signal of interest, block a signal of interest, etc., as desired. In various aspects of the device 300, properties of the electrodes 306 may be controllably variable. For example, at least one electrode of the device 300 may be set to be voltage, at least one electrode of the device 300 may be set to be current, and/or at least one electrode may be set to be an opposite current of the current. Examples of devices appropriate to such configuration are disclosed and described in U.S. provisional application Ser. No. 61/114,441; the disclosure of which is herein incorporated by reference.

### Method for Therapy Modulation

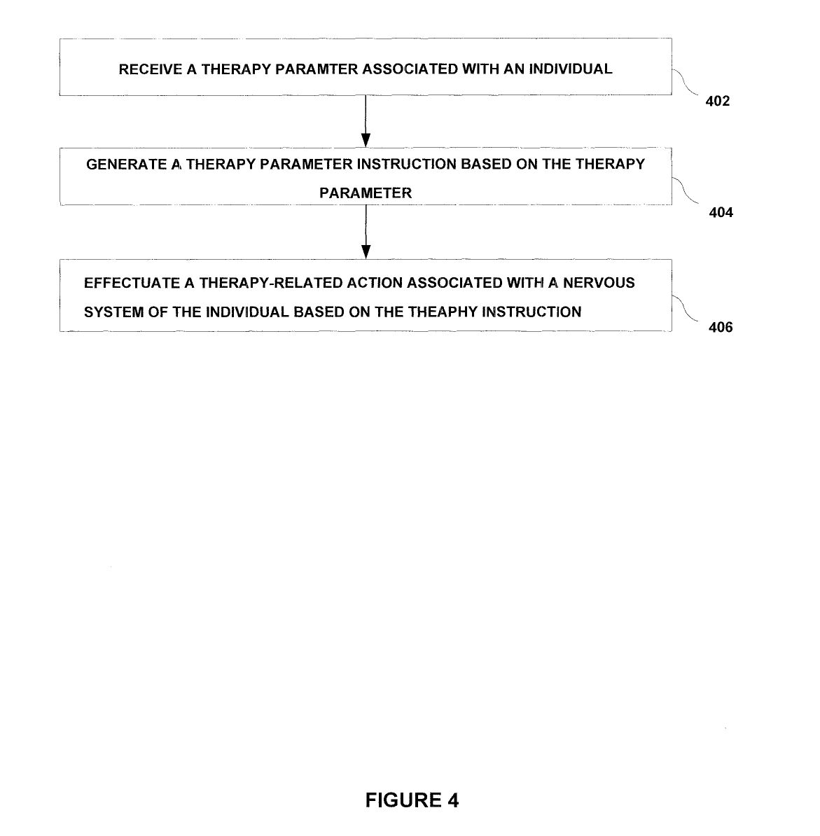

FIG. 4 illustrates a method for therapy modulation. In operation 402, a therapy parameter, e.g., the therapy parameter 202 of FIG. 2, associated with an individual, e.g., the patient 104, is received. In operation 404, a therapy instruction, e.g., the therapy instruction 204, is generated based on the therapy parameter. In operation 406, a therapy-related action, e.g., the therapy-related action 208, associated with a nervous system of the individual is effectuated based on the therapy instruction, which was triggered by the therapy parameter. Optionally, the method may include various other steps, e.g., sensing a therapy parameter using a sensor, e.g., a local or remote sensor; transmitting the therapy parameter, etc.

A skilled artisan will note that the aforementioned configurations are for illustrative purposes only and that various other components and configurations are possible. Communication-related modes include manual, wired, and wireless, etc. Wireless technologies include radio signals, such as x-rays, ultraviolet light, the visible spectrum, infrared, microwaves, and radio waves, etc. Wireless services include voice and messaging, handheld and other Internet-enabled devices, data networking, etc.

Vehicles of communication include the Internet, wired channels, wireless channels, communication devices including telephones, computers, wire, radio, optical or other electromagnetic channels, and combinations thereof, including other devices and/or components capable of/associated with communicating data. For example, the communication environments include in-body communications; various devices; various modes of communications such as wireless communications, wired communications, and combinations of the same, etc.

In-body communications include any communication of data or information via the body, i.e., communication via or associated with inter-body aspects, intra-body aspects, and a combination of the same. For example, inter-body aspects include communications associated with devices designed to attach to a body surface. Intra-body aspects include communications associated with data generated from within the body, e.g., by the body itself or by a device implanted, ingested, or otherwise locatable in, or partially in, the body.

Communications include and/or may be associated with software, hardware, circuitry, various devices, and combinations thereof. The devices include devices associated with therapy parameter, transmission, reception, communication, etc. The devices further include various implantable, ingestible, insertable, and/or attachable devices associated with the human body or other living organisms. The devices further include multimedia devices such as telephones, stereos, audio players, PDA's, handheld devices, and multimedia players.

Wireless communication modes include any mode of communication between points that utilizes, at least in part, wireless technology including various protocols and combinations of protocols associated with wireless transmission, data, and devices. The points include, for example, wireless devices such as wireless headsets; audio and multimedia devices and equipment, such as audio players and multimedia players;

telephones, including mobile telephones and cordless telephones; and computers and computer-related devices and components, such as printers.

Wired communication modes include any mode of communication between points that utilizes wired technology including various protocols and combinations of protocols associated with wired transmission, data, and devices. The points include, for example, devices such as audio and multimedia devices and equipment, such as audio players and multimedia players; telephones, including mobile telephones and cordless telephones; and computers and computer-related devices and components, such as printers.

Devices include, for example, any device capable of receiving, storing, and or transmitting the therapy parameters. Examples of such devices include a mobile telephone, personal digital assistant (PDA), computer, etc. Communication modes include any mode, channel, etc., capable of facilitating transmission, receipt, storage, etc. of the therapy parameter. Examples of communication modes include wired, wireless, etc.

One or more aspects of the subject invention may be in the form of computer readable media having programming stored thereon for implementing the various methods, or various steps thereof. The computer readable media may be, for example, in the form of a computer disk or CD, a floppy disc, a magnetic “hard card”, a server, or any other computer readable media capable of containing data or the like, stored electronically, magnetically, optically or by other means. Accordingly, stored programming embodying steps for carrying-out the subject methods may be transferred or communicated to a processor, e.g., by using a computer network, server, or other interface connection, e.g., the Internet, or other relay means.

It is to be understood that this invention is not limited to particular aspects described, and, as such, may vary. It is also to be understood that the terminology used herein is for the purpose of describing particular aspects only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference in their entirety as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual aspects described and illustrated herein has discrete components and features which may be readily separated from or combined with the features of any of the other several aspects without departing from the scope or spirit of the present invention. Any recited method can be carried out in the order of events recited or in any other order which is logically possible.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art and are to be construed as being without limitation to such specifically recited examples and conditions. Moreover, all statements herein reciting principles, aspects, and aspects of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary aspects shown and described herein. Rather, the scope and spirit of present invention is embodied by the appended claims.

1. A method of a sensing system for therapy modulation, comprising:
receiving a therapy parameter associated with an individual via a receive module of the sensing system; and
effectuating, based on the therapy parameter, a therapy-related action associated with a nervous system of the individual using a therapy-related action module of the sensing system.

2. The method of clam 1, wherein the receiving the therapy parameter further comprises generating a therapy parameter instruction based on the therapy parameter to perform the effectuating of the therapy-related action.

3. The method of claim 1, wherein the therapy parameter is selected from the group consisting essentially of a physiologic parameter, a subjective parameter, a chemical parameter, and an electrical parameter.

4. The method of claim 3, wherein the physiologic parameter is selected from the group consisting essentially of heart rate, heart rate variability, an edema indicator, a fluid retention indicator, temperature, a perspiration indicator, a galvanic skin response, blood pressure, and a vasodilatation indicator.

5. The method of claim 3, wherein the subjective parameter is selected from the group consisting essentially of a patient-perceived pain indicator, a patient-perceived tolerance indicator, and a patient-perceived optimal treatment indicator.

6. The method of claim 3, wherein the chemical parameter is selected from the group consisting essentially of extracellular signal-regulated kinase, gamma-aminobutyric acid, acetylcholine, and a neural activity indicator.

7. The method of claim 3, wherein the electrical parameter comprises an indicator of a conduction pattern associated with the nervous system.

8. The method of claim 1, wherein the therapy-related action is selected from the group consisting essentially of initiating a therapy, modifying the therapy, stopping the therapy, discontinuing the therapy, informing the individual of the therapy, tracking the therapy parameter, storing the therapy parameter, displaying the therapy parameter, generating a baseline indication associated with the individual, displaying the baseline indication, generating a historical data model including the therapy parameter, determining a program based on the baseline indication and the historical data model, analyzing the therapy data, automatically recommending the therapy based on the analysis of therapy data, automatically prompting the individual for feedback, and modifying the therapy based on the feedback.

9. A sensing system for therapy modulation, comprising:
a receive module to receive a therapy parameter associated with an individual; and
a therapy-related action module communicably coupled with the receive module, the therapy-related action module to effectuate, based on the therapy parameter, a therapy-related action associated with a nervous system of the individual.

10. The system of claim 9, wherein the therapy parameter is selected from the group consisting essentially of a physiologic parameter, a subjective parameter, a chemical parameter, and an electrical parameter.

11. The system of claim 9, further comprising a remote sensing module communicably coupled to the receive module to sense the therapy parameter.

12. The system of claim 11, wherein the remote sensing module is a cardiac sensing device, a fluid sensor, a temperature sensor, a chemical sensor, or an electrical sensor.

13. The system of claim 11, wherein the subjective parameter is communicated to the remote sensing module via a communication device of the individual.

14. The system of claim 10, wherein the subjective parameter is selected from the group consisting essentially of a patient-perceived pain indicator, a patient-perceived tolerance indicator, and a patient perceived-optimal treatment indicator.

15. The system of claim 9, wherein the receive module is configured to generate a therapy parameter instruction used to trigger the therapy related action based on the therapy parameter.

16. A device for therapy modulation, comprising:
at least one sensor to sense at least one therapy parameter of an individual; and
control logic circuitry in electrical communication with the at least one sensor to receive the at least one therapy parameter and to effect, based on the at least one therapy parameter, a therapy-related action associated with the individual.

17. The device of claim 16, wherein the device is an implanted device, a neural device, an extra-corporeal device, or a wearable device.

18. The device of claim 16, further comprising at least one electrode electrically coupled to the control logic circuitry configured to stimulate a tissue region of the individual.

19. The device of claim 16, wherein the control logic circuitry is implemented on a lead.

20. The device of claim 16, wherein the at least one sensor comprises a network of electrodes.
